(19) United States
(12) Patent Application Publication
(10) Pub. No.: US 2003/0199914 A1
(43) Pub. Date: Oct. 23, 2003

(54) Coaxial balloon catheter

(76) Inventor: Juan-Carlos Diaz, Miami, FL (US)

Correspondence Address: MEDTRONIC AVE, INC., 3576 UNOCAL PLACE, SANTA ROSA, CA, 95403, US

(21) Appl. No.: 10/128,977

(22) Filed: Apr. 23, 2002

(51) Int. Cl. A61M 29/00

(52) U.S. Cl. 606/194

(57) ABSTRACT

A coaxial balloon catheter (50) includes an elongated inner tube (14), an elongated outer tube (16) residing on the exterior of the elongated inner tube, and a balloon (20) having a distal end (22) attached to a first portion of the elongated outer tube and having a proximal end (21) attached to a second portion of the elongated outer tube. This arrangement prevents the balloon from telescopingly buckling when being pushed across a narrow passage.

### FIELD OF THE INVENTION

This invention relates generally to catheters, and more particularly to a coaxial balloon catheter arrangement.

### BACKGROUND OF THE INVENTION

Various forms of balloon catheters are used in medical procedures that could use additional improvements in terms of cross-sectional area, inflation/deflation times and columnar strength. Such medical procedures could include percutaneous transluminal coronary angioplasty (PTCA) and endovascular exclusion of abdominal aortic aneurysm (AAA) using a stent-graft deployment system.

PTCA is a procedure by which a balloon catheter is inserted into and manipulated within a patient's coronary arteries to unblock an obstruction (or a stenosis) in the artery. Typically, the catheter is about 150 cm long and is inserted percutaneously into the patient's femoral artery in the region of the groin. The catheter then is advanced upwardly through the patient's arteries to the heart where, with the aid of a guidewire, the catheter is guided into the coronary artery where it can be controlled to perform the angioplasty procedure.

In one type of PTCA catheter, the catheter has two lumens. One lumen, for inflation and deflation of the balloon, extends from a fitting at the proximal end of the catheter and opens distally into the interior of the balloon. The balloon is inflated with a liquid and is deflated by aspirating the liquid from the balloon through the inflation/deflation lumen. The second lumen extends from another fitting at the proximal end of the catheter through the catheter and is open at the distal tip of the catheter shaft. The second lumen is adapted to receive a guidewire, such as the steerable small diameter type of guidewire.

In a typical procedure, the guidewire is preliminarily loaded into the catheter and the assembly is inserted into a previously percutaneously placed guide catheter that extends to the region of the patient's heart and terminates at the entrance to the coronary arteries. The assembly of the balloon angioplasty catheter and the steerable guidewire is advanced through the guide catheter to the entrance to the coronary arteries. The guidewire then is projected into the coronary arteries and is steered by manipulation from its proximal end, while being observed under a fluoroscope, until the guidewire passes through the stenosis in the artery. Once the guidewire is in place, the balloon dilatation catheter is advanced over the guidewire, being thus guided directly to the stenosis so as to place the balloon within the stenosis. Once so placed, the balloon is inflated under substantial pressure to dilate the stenosis.

In the process of endoluminal AAA repair using a stent-graft deployment system, a balloon catheter could be used to appropriately seat the graft in a target area. In general, the use of stents, and stent-grafts for treatment or isolation of vascular aneurysms and vessel walls which have been thinned or thickened by disease (endoluminal repair or exclusion) are well known. Many stents and stent-grafts, are “self-expanding”, i.e., inserted into the vascular system in a compressed or contracted state, and permitted to expand upon removal of a restraint. Self-expanding stents typically employ a wire of suitable material, such as a stainless steel, configured (e.g. bent) to provide an outward radial force, and/or formed of shape memory wire such as Nitinol (nickel-titanium) wire. When the shape memory wire is employed, the stent is typically of a tubular configuration of a slightly greater diameter than the diameter of the blood vessel in which the stent is intended to be used. The stent is preferably treated to enable it to “remember” its initial configuration. In general, stents and stent-grafts are preferably deployed through a minimally invasive percutaneous intraluminal delivery as described with respect to the PTCA catheter. The stent-graft is routed through the vascular lumen to the site where the prosthesis is to be deployed. Intraluminal deployment is typically effected using a delivery catheter with coaxial inner (plunger) and outer (sheath) tubes arranged for relative axial movement. The stent to be deployed is compressed and disposed within the distal end of an outer catheter tube in front of an inner tube. The catheter is then maneuvered, typically routed though a lumen (e.g., vessel), until the end of the catheter (and the stent or stent-graft) is positioned in the vicinity of the intended treatment site. The inner tube is then held stationary when the outer tube of the delivery catheter is withdrawn. The inner tube prevents the stent-graft from being withdrawn with the outer tube, so that, as the outer tube is withdrawn, the stent radially expands into a substantially conforming surface in contact with the interior of the lumen e.g., blood vessel wall. To avoid “endoleaks”, a balloon on a balloon catheter can be used to appropriately seat the stent-graft to the blood vessel wall or walls.

In any event, to avoid additional trauma in such procedures as described above, it is preferable to minimize the amount of time needed to utilize the balloon, minimize the cross-sectional area of the catheter, and to provide as much control to the operating physician as possible. One way to achieve reduced utilization time and provide greater control involves reducing the inflation and deflation times needed. Another way to provide additional control includes having adequate columnar strength. Yet another way is to provide a centered guidewire lumen rather than an offset guidewire lumen. Multi-lumen (non-coaxial or side by side) catheters will usually have poorer performance in providing control as described above in comparison to comparable coaxial catheters, particularly in terms of reduced cross-sectional area and inflation/deflation times. Furthermore, multi-lumen catheters have guidewire lumens that are offset by the inflation lumens.

The anatomy of coronary arteries varies widely from patient to patient. Often a patient's coronary arteries are irregularly shaped and highly tortuous. The tortuous configuration of the arteries may present difficulties to the physician in properly placing the guidewire and then advancing the catheter over the guidewire or seating a stent or stent-graft. A highly tortuous coronary anatomy typically will present considerable resistance to advancement of the catheter over the guidewire. With some types of catheter construction, the increased resistance may cause a tendency for portions of the catheter to collapse or buckle axially. For example, in a catheter having a shaft formed from inner and outer coaxial tubes and a balloon mounted to the distal ends of the tubes, there may be a tendency for the tubes to move axially relative to one another (telescope) when the assembly encounters an increased resistance. The telescoping of the tubes will tend to push the ends of the balloon together slightly but sufficiently to permit the balloon to become bunched up as it is forced through the stenosis. The bunching up of the balloon makes it more difficult for the balloon to cross the stenosis. Thus, a need exists for a balloon catheter that overcomes the detriments described above and provides for reduced inflation/deflation times and increased control.

### SUMMARY OF THE INVENTION

In a first aspect according to the present invention, a coaxial balloon catheter includes an elongated inner body, an elongated outer body residing on the exterior of the elongated inner body, and a balloon having a distal end attached to a first portion of the elongated outer body. A proximal end is attached to a second portion of the elongated outer body. In a second aspect according to the present invention, a balloon dilatation catheter includes an elongate, flexible catheter shaft including a two tube, two lumen configuration. The shaft having a proximal region, a proximal end and a distal end. The shaft is formed from an inner tube defining a guidewire lumen therethrough, and a surrounding outer tube coaxial with the inner tube and defining an annular inflation lumen therebetween. The inner tube being of smaller diameter than the outer tube and having a distal end region extending distally of the distal end of the outer tube. The balloon dilatation catheter further includes an inflatable dilatation balloon having a proximal end and a distal end. The proximal end of the balloon being attached to a first distal region of the outer tube. The distal end of the balloon being attached to a second distal region of the outer tube at a distal connection. The distal end is connected to means for communicating the annular inflation lumen with the interior of the balloon to facilitate inflation and deflation of the balloon. The inflation lumen is the sole lumen in communication with the interior of the balloon. The catheter also includes means at the proximal end of the catheter for accessing each of the guidewire and the inflation lumens. In yet another aspect according to the present invention, a method of manufacturing a coaxial balloon catheter includes the steps of (1) inserting an elongated inner body coaxially within an elongated outer body, (2) forming at least one aperture on the elongated outer body to serve as an inflation port, (3) attaching a distal end of a balloon adjacent to a distal region of the elongated outer body, (4) attaching the distal region of the elongated outer body to an outer portion of the elongated inner body, and (5) attaching a proximal end of the balloon on a portion of the elongated outer body, wherein the balloon covers the at least one aperture.

### BRIEF DESCRIPTION OF THE DRAWINGS

FIG. 1 is an illustration of a catheter of the type with which an embodiment according to the invention can be deployed.

FIG. 2 is an enlarged partial cross-sectional view of a portion of an elongated inner body centered within an elongated outer body in a configuration according to the present invention.

FIG. 3 is another enlarged partial cross-sectional view of an internal assembly of the elongated inner body and elongated outer body of FIG. 2 showing the outer body attached to the inner body and an inflation/deflation port in the outer body, the balloon not being shown.

FIG. 4 is an enlarged partial cross-sectional view of a portion of the balloon catheter having both proximal and distal ends of a balloon attached to the outer body in accordance with the present invention.

FIG. 5 is an alternative embodiment of FIG. 4.

FIG. 6 is a cross-sectional view taken at 6-6 of FIG. 2 illustrating the construction and configuration of the inner and outer tubes of the catheter.

FIG. 7 is a cross-sectional view taken at 7-7 of FIG. 4 illustrating the construction and configuration of the balloon and the inner and outer tubes of the catheter.

### DETAILED DESCRIPTION

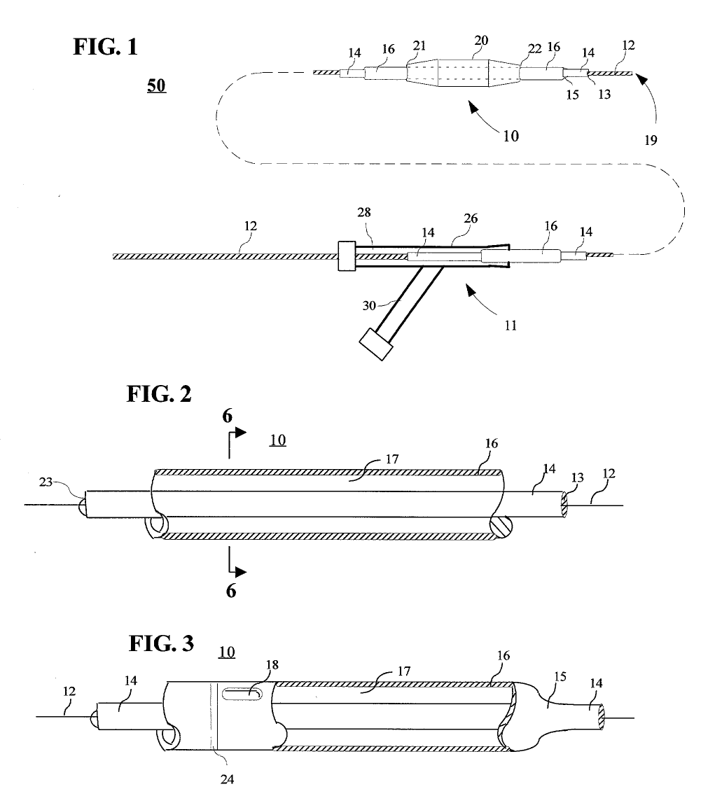

As shown in FIG. 1, the catheter 50 includes a distal assembly 10 of the catheter. The catheter 50 has a proximal end 11 and a distal end 19. A dilatation balloon 20 is mounted near the distal end of the assembly 10. In an embodiment according to the invention, the assembly 10 includes a pair of coaxial tubes 14, 16 illustrated partly in enlarged detail in FIGS. 2-6. FIGS. 2-6 further illustrate a process to manufacture a catheter in accordance with the present invention, as will be further discussed below. The coaxial tubes include an inner tube 14 and an outer tube 16. The tubes 14, 16 may be polyethylene, with the inner tube, for example, being a high density polyethylene and the outer tube being a linear low density polyethylene. It should be understood that the outer and inner tubes can be formed of any known material suitable for similar medical devices including such materials as polyamide, polyurethane, polyester, peek, nylon, and polyethylene or any combination thereof. For instance, in one embodiment as shown in FIG. 6, the outer tube 16 can be formed of an outer layer 51 of polyamide or polyurethane and an inner layer 52 (of the outer tube 16) can be formed of nylon material, wherein the polyamide would provide better bonding characteristics with other plastics (such as with the balloon 20) and the inner layer would provide added columnar strength. By way of example, the catheter may be approximately 110 cm to 155 cm long depending on the specific application. The inner tube may have an outside diameter of about 0.054″ and an inside diameter of 0.042″, the wall thickness being approximately 0.006″. The outer tube 16 may have an outside diameter of about 0.106″ and an inside diameter of the order of 0.084″ with a wall thickness of about approximately 0.011″. The inner tube 14 defines an inner lumen 23 adapted to receive a guidewire 12 (FIG. 1) with the proximal and distal ends of the guidewire 12 extending beyond the proximal and distal ends of the catheter 50. The inner tube 14 extends fully to the distal tip 13 (see FIG. 2) of the catheter. An annular inflation lumen 17 is defined between the inner tube 14 and the outer tube 16.

The proximal end 11 of the catheter is provided with a Y-fitting 26 which may be molded from an appropriate plastic and to which is connected a pair of proximal tubes 28, 30 as known in the art. The Y-fitting 26 is formed so that the proximal tube 28 is in communication with the guidewire lumen 23 in the inner tube 14 and the proximal tube 30 is in communication with the annular inflation lumen 17. Each of the proximal tubes 28, 30 is provided with a fitting at their respective proximal ends by which a guidewire or appropriate fluid handling devices such as syringes, inflation devices or the like may be connected.

The guidewire lumen 23 extends from the proximal end 11 to the distal end 19 of the catheter and terminates in an outlet opening 13. Thus, the guidewire 12, which is much longer than the catheter may have the catheter passed over the guidewire 12 via the guidewire lumen 23 and may exit from the outlet tip 13, with the proximal end of the guidewire 12 protruding proximally from the proximal tube 28. The guidewire may be manipulated from its proximal end and may be steered through the coronary anatomy to the branch of the coronary arteries where the stenosis or aneurysm is located.

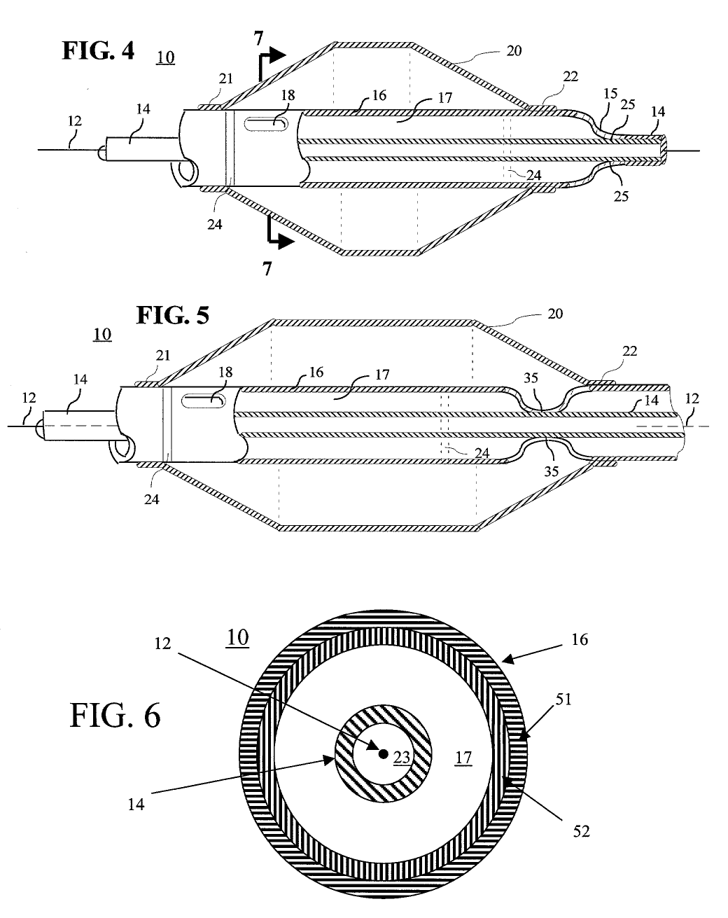

The outer tube 16 extends from the Y-fitting 26 to a location short of the end of the inner tube 14 and extends beyond the balloon 20. It should be understood that even though the outer tube extends beyond the balloon 20, a bond or seal 25 between the outer tube 16 and the inner tube 14 to form the end seal for the balloon inflation lumen 17 could be located within or beyond the balloon 20 at a location that does not necessarily correspond to the distal end of the outer tube 16. Preferably, though, in accordance with the invention, and as described further below, the distal end 15 of the outer tube 16 is securely attached or anchored (preferably bonded, fused or sealed) to the inner tube 14 at a location adjacent to the distal end of the balloon 20. The outer tube 16 and the inner tube 14 may be secured to each other by an appropriate adhesive (e.g., ultraviolet cured urethane adhesives, cyanoacrylate, epoxy) or by heat bonding or fusing the inner and outer tubes together. FIG. 4 shows the bond 25 between the outer tube 16 and the inner tube 14 just beyond the distal end of the balloon 20. Alternative embodiment (FIG.) 5 shows the bond 35 between the outer tube 16 and the inner tube 14 between the proximal and distal ends of the balloon 20, but note that outer tube continues past the bond and beyond the end of the balloon 20.

Referring to FIGS. 3 and 4, at least one aperture 18 is formed in the outer tube 16 within the proximal and distal seal limits of the balloon 20 to provide a passage between the inflation lumen 17 and the interior of the balloon so as to permit inflation and deflation of the balloon with an appropriate liquid such as saline, or contrast, or others as will be familiar to those skilled in the art. The juncture, e.g., end of the outer tube, 15 at which the outer tube 16 is bonded (or fused) 25 and sealed to the inner tube 14 as well as the size and number of apertures (one is shown, but more could be provided) (18) defines the inflation lumen 17 and provides the catheter with a improved mechanism to reduce inflation and deflation times. This gives a physician the ability to further reduce the amount of time a patient is under the invasive process of catheterization.

A first portion or a first distal region of the outer tube 16 is preferably coupled (adhesively attached, bonded, fused, sealed, or otherwise) to a proximal neck 21 of the balloon 20. The distal end of the balloon is provided with a cylindrical distal neck 22 which is coupled (once again adhesively attached, bonded, fused, sealed or otherwise) a second portion or a second distal region of the outer tube 16. The balloon typically could include proximal and distal cone sections and a central cylindrical section, as will be appreciated by those skilled in the art. The balloon may be formed from a suitable material such as polyethylene terephthalate. It may be made in a manner described in U.S. Pat. No. 4,490,421 (Levy). The balloon may be adhesively attached to the two portions of the outer tube by suitable adhesive such as an ultraviolet cured urethane adhesive.

The catheter may be provided with a small band 24 of highly radiopaque material such as gold, about the inner tube 14 or the outer tube 16 (as shown) within the region of the balloon in order to render the balloon region of the catheter visible under fluoroscopy. Byway of example, the marker band 24 may be approximately 1 mm long and may have a wall thickness of about 0.002″. It is retained in place on the inner or outer tube by a heat shrunk encapsulating tube of an appropriate plastic, such as a linear low polyethylene material.

From the foregoing, it will be appreciated that after the guidewire has been desirably placed in the patient's coronary anatomy, the physician will then advance the catheter over and axially along the guidewire. Should the coronary anatomy present resistance, as by presenting a narrow difficult stenosis and/or tortuous path, the increased column strength resulting from anchoring and preferably bonding or fusing the distal end of the outer tube 16 to the inner tube 14 will increase the pushability of the catheter. The axial force applied to both the inner and outer tubes is available to push the catheter through the tortuous anatomy and/or the balloon through the difficult stenosis. With the foregoing arrangement, the tendency of the inner tube to telescope, buckle or collapse is avoided. Because the balloon is bonded only to the outer tube 16, the axial distance between the ends of the balloon is maintained and the balloon will not bunch up as it is pushed through a tight stenosis.

Referring once again to FIGS. 2-4, a method of manufacturing a coaxial balloon catheter is illustrated. In FIG. 2, an elongated inner tube 14 is coaxially inserted within an elongated outer tube 16. In FIG. 3, at least one aperture 18 is formed on the elongated outer body to serve as an inflation or deflation port. A cut-out portion of the elongated outer body provides the port. Also in FIG. 3, the elongated outer body is fused or bonded to the outer portion of the elongated inner body. As previously explained, the bond or fusing of the outer body 14 to the inner body can be located along the catheter between the proximal and distal ends of the balloon as shown in FIG. 5 or beyond the distal end of the balloon as shown in FIG. 4. As shown in FIG. 4, the distal end of a balloon is fused or bonded adjacent to the distal end of the elongated outer body and the proximal end of the balloon is fused or bonded on a portion of the elongated outer body, wherein the balloon encloses the aperture. The method could also include the step of placing radiopaque marker bands 24 (as shown) adjacent to the proximal and distal ends of the balloon. These marker bands could be on either the inner or outer bodies within the balloon.

The invention thus provides an improved coaxial catheter construction for a catheter by which the column strength and resistance to telescopic buckling of the catheter, and particularly, of the inner tube and balloon of a coaxial catheter, is improved. The resulting catheter has increased pushability. Bunching up of the balloon is avoided. Additional benefits include reduced inflation/deflation times with a coaxial design giving a centered guidewire lumen providing additional control and placement accuracy for the operating physician. The coaxial design can further be arranged and constructed to have a reduced cross-sectional area (relative to non-coaxial designs) that further minimizes trauma during catheterization.

Referring to FIG. 5, an alternative embodiment of FIG. 4 is shown. Similar to the embodiment of FIG. 4, the portion of the balloon catheter shown includes a distal end 15 of an outer tube 16 that is securely attached or anchored (preferably bonded, fused or sealed) to an inner tube 14 at a location adjacent to the distal end of the balloon 20. The outer tube 16 and the inner tube 14 may be secured to each other as previously described with regard to the embodiment of FIG. 4. FIG. 5 illustrates a bond 35 between the outer tube 16 and the inner tube 14 between the proximal and distal ends of a balloon 20, but note that outer tube continues past the bond and beyond the end of the balloon 20.

Referring to FIG. 6, a cross-sectional view taken at 6-6 of FIG. 2 illustrates the construction and configuration of the inner and outer tubes of the catheter in further detail. FIG. 6 illustrates the annular inflation lumen 17 defined by the volume between the inner tube 14 and the outer tube 16 as well as the guidewire lumen defined by the volume within inner tube 14. The outer tube 16 can be formed of an outer layer 51 of polyamide or polyurethane and an inner layer 52 (of the outer tube 16) can be formed of nylon material, wherein the polyamide would provide better bonding characteristics with other plastics (such as with the balloon 20) and the inner layer would provide added columnar strength.

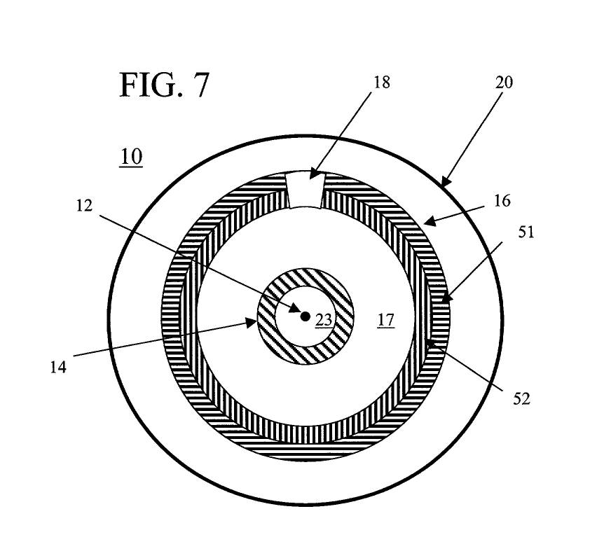

Referring to FIG. 7, a cross-sectional view taken at 7-7 of FIG. 4 illustrates the construction and configuration of the balloon 20 and the inner and outer tubes of the catheter in further detail as previously explained above with regard to FIG. 6. In addition, FIG. 7 illustrates the cross-section view of the aperture 18 formed in the outer tube 16. The aperture 18 serves as the inflation/deflation port for inflating or deflating the balloon 20 via inflation lumen 17.

It should be understood, however, that the foregoing invention is intended merely to be illustrative thereof and that other embodiments and modifications may be apparent to those skilled in the art without departing from its spirit and scope.

What is claim is:

1. A coaxial balloon catheter, comprising:
an elongated inner body;
an elongated outer body surrounding a portion of said elongated inner body;
a balloon having a proximal end attached to a first portion of the elongated outer body and having a distal end attached to a second portion of the elongated outer body.

2. The coaxial balloon catheter of claim 1, wherein the elongated outer body has an inflation/deflation port formed therein at a location between the distal end and the proximal end of the balloon.

3. The coaxial balloon catheter of claim 1, further comprising marker bands located within the balloon near opposing ends of the balloon.

4. The coaxial balloon catheter of claim 1, wherein the inner body includes a guide wire lumen.

5. The coaxial balloon catheter of claim 1, wherein a portion of the elongated outer body is attached to a portion of the elongated inner body.

6. The coaxial balloon catheter of claim 1, wherein the distal end and proximal end of the balloon are attached to the first portion and the second portion respectively of the elongated outer body by one of the attachment methods selected from the group of bonding, fusing or sealing.

7. The coaxial balloon catheter of claim 5, wherein the portion of the elongated outer body attached to the portion of the elongated inner body is a distal end of the elongated outer body and wherein the elongated outer body is attached to the elongated inner body by one of the attachment methods selected from the group of bonding, fusing or sealing.

8. A balloon dilatation catheter comprising:
an elongate, flexible catheter shaft including a two tube, two lumen configuration, the shaft having a proximal region, a proximal end and a distal end, wherein the shaft is formed from an inner tube defining a guidewire lumen therethrough, and a surrounding outer tube coaxial with the inner tube and defining an annular inflation lumen therebetween, the inner tube being of smaller diameter than the outer tube;
an inflatable dilatation balloon having a proximal end and a distal end, the proximal end of the balloon being attached to a first distal region of the outer tube, the distal end of the balloon being attached to a second distal region of the outer tube at a distal connection;
means for communicating the annular inflation lumen with the interior of the balloon to facilitate inflation and deflation of the balloon, the inflation lumen comprising the sole lumen in communication with the interior of the balloon; and
the outer tube being attached to the inner tube at a location between the means for communicating and the distal end region of the inner tube.

9. A coaxial balloon dilatation catheter as in claim 8 wherein the distal end of the outer tube is attached to the inner tube at a location within the inflatable portion of the balloon.

10. A coaxial balloon dilatation catheter as defined in claim 8 wherein the distal end of the outer tube is attached to the inner tube at a distal location on the outer tube that is outside the inflatable portion of the balloon.

11. A catheter as defined in claim 9 wherein the means communicating the inflation lumen with the interior of the balloon comprises at least one aperture serving as an inflation port associated with the outer tube for communicating the inflation lumen with the interior of the balloon.

12. A catheter as defined in claim 11 wherein a portion of the outer tube is disposed within the balloon and wherein the at least one aperture is formed in the portion of the outer tube that is disposed within the balloon.

13. The balloon dilatation catheter as defined in claim 8, further comprising means at the proximal end of the catheter for accessing each of the guidewire and inflation lumens.

14. The balloon dilatation catheter of claim 8, wherein the inner tube and the outer tube are formed from materials selected from the group comprising polyethylene, polyester, polyurethane, polyamide, peek, nylon, or any combination thereof.

15. The balloon dilatation catheter of claim 8, wherein the outer tube is attached to the inner tube.

16. The balloon dilatation catheter of claim 8, wherein the outer tube is attached to the inner tube to form a seal to create the annular inflation lumen.

17. A method of manufacturing a coaxial balloon catheter, comprising the steps of:
forming at least one aperture on an elongated outer body to serve as an inflation port;
inserting the elongated inner body coaxially within an elongated outer body;
attaching a distal end of a balloon adjacent to a distal region of the elongated outer body;
attaching the distal region of the elongated outer body to an outer portion of the elongated inner body; and
attaching a proximal end of the balloon on a portion of the elongated outer body, wherein the balloon covers the at least one aperture.

18. The method of claim 17, wherein the method further comprises the step of placing radiopaque marker bands adjacent to the proximal and distal ends of the balloon.

19. The method of claim 17, wherein the step of forming an aperture comprises the step of cutting-out a portion of the elongated outer body.

20. The method of claim 17, wherein each step of attaching comprises the step selected from the group of bonding, sealing, or fusing.
